(19) United States
(12) Patent Application Publication
(10) Pub. No.: US 2019/0336674 A1
(43) Pub. Date: Nov. 7, 2019

(54) DEVICE AND METHOD FOR AUTOMATICALLY VENTING AND FILLING A CATHETER

(71) Applicant: FRESENIUS MEDICAL CARE DEUTSCHLAND GMBH, Bad Homburg (DE)

(72) Inventors: Olaf SCHERMEIER, Frankfurt (DE); Kirill KOULECHOV, Bad Vilbel (DE)

(73) Assignee: FRESENIUS MEDICAL CARE DEUTSCHLAND GMBH, Bad Homburg (DE)

(21) Appl. No.: 16/479,938

(22) PCT Filed: Jan. 29, 2018
(86) PCT No.: PCT/EP2018/052151

§ 371 Date: Jul. 23, 2019

(30) Foreign Application Priority Data
Jan. 30, 2017 (DE) 10 2017 201 439.3

(51) Int. Cl. A61M 1/36

(52) U.S. Cl. CPC A61M 1/3643; A61M 2205/331; A61M 2025/1077; A61M 2205/35; A61M 2205/50; A61M 2205/3331

(57) ABSTRACT

The invention relates to an apparatus and a method for the automatic deaerating and filling of a catheter connected to a blood vessel of a patient with blood, in particular in preparation for hemodialysis, and in particularly by means of a cannulation robot. The invention implements this in particular by the deaerating and filling of a fluid channel fluidly connected to the catheter by means of a deaeration device, by the detecting of the deaerating and/or filling of the fluid channel by a sensor device which generates a measurement signal containing information on the deaerating and/or filling of the fluid channel, and by the controlling of the deaeration device as a function of the measure-ment signal by means of a control device with electrical circuitry so as to achieve the automated deaerating and/or filling of the fluid channel and the catheter.

The present invention relates to an apparatus and a method for the automatic deaerating and filling of a catheter connected to a blood vessel of a patient with blood, in particular in preparation for hemodialysis.

Deaerating a catheter is in particular necessary when the blood withdrawn through the catheter is to thereafter be returned back into the patient's blood circulation free of air bubbles. A typical application is hemodialysis. The deaerating serves to minimize the risk of an aeroembolism. A critical limit of air volume introduced into the blood vessel is between 10 to 25 ml. Similar and greater volumes of air are regarded as particularly risky with respect to the development of a life-threatening aeroembolism.

Hemodialysis is contemplated in cases of chronic kidney diseases in which the kidney's blood cleansing function is replaced by dialysis equipment. Such dialyses are usually performed manually at the present time and require relatively high expenditure in terms of material, time and care. In extracorporeal hemodialysis, the blood is extracted from the body at a first puncture site of a blood vessel, purified in the extracorporeal blood circulation of the dialysis equipment, and returned to the intracorporeal blood circulation at a second puncture site of a blood vessel. Providing a reliable connection of the extracorporeal and intracorporeal blood circulation without the formation of air bubbles in the circulation in particular requires a plurality of steps.

The present invention is based on the task of providing an efficient apparatus and an efficient method for achieving automatic deaerating and filling of a catheter connected to a blood vessel of a patient with blood, in particular in preparation for hemodialysis.

The invention solves this task by means of an apparatus in accordance with the teaching of independent claim 1 and a method in accordance with the teaching of independent claim 17.

Preferential embodiments, further developments or variants in particular constitute the subject matter of the independent claims.
The apparatus according to the invention for the automatic deaerating and filling of a catheter connected to a patient's blood vessel with blood, in particular in preparation for hemodialysis, comprises: a fluid channel section for guiding fluid flows and a deaeration device for deaerating the catheter, wherein the fluid channel section comprises: at least one fluid channel through which fluids can flow, a blood inflow channel configured for catheter attachment and for a fluid connection to the fluid channel, an air outflow channel configured for a fluid connection to the fluid channel and the deaeration device such that air can be conveyed out of the fluid channel and out of the catheter connected to the blood inflow channel through the air outflow channel by means of the deaeration device, whereby the catheter connected to the blood vessel and the fluid channel can be filled with blood, wherein the apparatus comprises a sensor device designed to measure the deaerating and/or filling of the fluid channel and to generate a measurement signal containing information on the deaerating and/or filling of the fluid channel, and wherein the apparatus comprises a control device with an electrical circuitry configured to automatically control the deaeration device for deaerating and/or filling the fluid channel as a function of the measurement signal.

The measuring of the deaerating and/or filling of the fluid channel enables achieving a reliable automating of the deaerating and/or filling of said fluid channel. This can thereby particularly reliably minimize the inclusion of the air found within the catheter and the fluid channel prior to the deaeration into the extracted blood.

The sensor device preferably comprises an optical sensor and in particular a measuring zone provided in the fluid channel. Blood entering into the measuring zone can be detected by means of a change in an optical parameter, in particular the color and/or brightness, while the optical sensor can detect continuous measurements or measurements performed at periodic intervals. The optical sensor can comprise a photodetector. A photodetector is an electronic element which uses the photoelectric effect to convert light into an electrical signal or exhibits an electrical resistance dependent on the incident radiation. Preferably, the fluid channel section comprises a transparent sensor section via which the light emitted from the measuring zone can enter and can be measured by the optical sensor preferably arranged external of the fluid channel. The sensor device can also comprise a light source for radiating light into the measuring zone under the influence of the measuring zone and/or as a function of the fluid disposed in the measuring zone, in particular transmitted, diffracted, scattered and/or or reflected. Optical measurement enables a particularly reliable measuring of the deaerating and/or filling of the fluid channel.

The sensor device preferably comprises a pressure sensor and in particular a pressure absorber which is in particular fluidly connected to the fluid channel in a contact area of the fluid channel. The pressure sensor can directly detect blood entering into the contact area by means of a change in pressure during continuous measurements or measurements performed at periodic intervals. The pressure absorber preferably comprises a flexible membrane which is mechanically deformed by a change in pressure. This deformation is measured by an mechanical/electrical converter, in particular a piezoelectric element, and the output measurement signal is electrical. The sensor section is preferably the flexible membrane. A pressure measurement in particular enables measuring the deaerating and/or filling of the fluid channel.

The sensor device can exhibit exactly one sensor or a plurality of sensors. By arranging multiple sensors along the fluid channel's direction of flow, particularly the intake progression of the blood can be determined, in particular differentiated over time, with an inflow velocity in particular being able to be measured. This and/or other information can be used by the control device to precisely determine the blood intake into the fluid channel. In particular, the control device can stop the filling of the fluid channel by means of the venting device when the blood reaches the first sensor and/or reaches the second sensor or when the blood reaches the first sensor and has not yet reached the second sensor. The first sensor can be arranged adjacent to a blood outflow channel and the second sensor can be arranged adjacent to the air outflow channel, in particular in order for the control device to be able to achieve the conduction of the blood into the blood outflow channel without any air bubbles and no blood being able to intrude into the deaeration device, which is particularly desired when the deaeration device is realized as a vacuum pump.

The measurement signal can be analog or digital. When the sensor device outputs measurement signal data, the measuring measurement can in particular contain measurement signal data.

Electrical circuitry of the sensor device and/or the control device can analyze the measurement signal. The control device is in particular designed to evaluate the measurement signal. The control device can be designed to compare the measurement signal data obtained from the measurement signal to reference data and, as a function of said reference data, output the result of a filling of the fluid channel with blood being determined and/or the result of a filling of the fluid channel with blood not being determined.

The control device can comprise a control program executable by a data processor, in particular a CPU, for controlling the deaeration device and/or at least one valve of the apparatus and can contain an analysis program and be designed to execute said analysis program.

The fluid channel section can comprise a housing in which at least the fluid channel is arranged. At least one blood inflow channel can respectively open into another catheter connector positioned external of the housing. At least one blood outflow channel can respectively open into another line connector positioned external of the housing. The air outlet channel can open into a line connector positioned external of the housing. At least one air outlet channel can respectively open into another line connector positioned external of the housing. The deaeration device and/or at least one sensor device and/or at least one valve can be arranged in the housing. The fluid channel section, in particular its housing, preferably consists of a polymeric compound and preferably comprises at least one or two pieces, in particular exactly two, which are produced via injection molding and in particular joined together.

A disposable item in the context of the present invention is in particular characterized by the fact of preferably consisting of at least one polymer. This does not apply to a cannula which preferably consists substantially of metal.

The catheter connector, in particular also the line connector, can in particular be configured to establish a form-fit and/or force-fit connection of the catheter/line to the catheter/line connector, can in particular be designed for interlocking, and can in particular be designed as a luer lock connection. A luer lock connection is a technically standardized connection.

Preferably, the apparatus comprises a blood outflow channel which is designed to deliver the blood from the fluid channel to an extracorporeal blood guidance system.

Preferably, the fluid channel section, in particular the fluid channel, comprises a sensor section designed to detect the entry of blood into the fluid channel by means of the sensor device.

Preferably, the sensor device and/or the deaeration device is/are a part of the fluid channel section.

The apparatus preferably comprises a (single) fluid channel section, however can also comprise two or more identically or differently configured fluid channel sections.

The fluid channel section serves in particular as a distancing spacer arranged between the catheter and the deaeration device or, respectively, the catheter and the flushing device. The spacing reduces the risk of the catheter being contaminated and preserves the sterility.

Preferably, the fluid channel section comprises a rinsing inflow channel which is designed for the supplying of and the flowing of a physiological rinsing fluid through said fluid channel. The rinsing fluid is preferably a saline solution and can in particular contain a pharmaceutical, e.g. an active anticoagulation substance, in particular heparin. The salt concentrations of the saline solution preferably correspond at least partly to the typical physiological salt concentrations in blood plasma. The—preferably one-time or repeated—flushing of the fluid channel and the catheter acts to inhibit thrombosis since the blood coming into contact with the catheter and the fluid channel during deaerating can give rise to a risk of thrombosis which the rinsing process reduces. The flushing therefore achieves a reliable connection of the intracorporeal and extracorporeal blood circulation, particularly in preparation for hemodialysis. The fluid channel section comprising the air outlet channel and the rinsing inflow channel also enables omitting the step necessary when manually priming the catheter of first needing to connect the catheter to a vacuum pump, then needing to uncouple same again once the catheter hose clip is closed and needing to couple the rinsing fluid feed. In particular, the at least one valve preferably provided in the fluid channel section serves to functionally replace a catheter hose clip. Integrating the different functions into the fluid channel section achieves a high efficiency and reliability in the automated operation of the fluid channel section and the automated priming.

Preferably, the apparatus, in particular the fluid channel section, comprises a conveyor device, in particular a pump, for pumping the physiological rinsing fluid. Preferably, the control device is designed to control the conveyor device such that the physiological rinsing fluid is moved through the fluid channel and the catheter by means of the conveyor device.

The physiological rinsing fluid can in particular be moved through the fluid channel and the catheter by means of the deaeration device. Preferably, the control device is designed to control the deaeration device such that the physiological rinsing fluid is moved through the fluid channel and the catheter by means of the deaeration device. This can be achieved by the deaeration device being operated in the opposite direction of conveyance relative to the prevailing direction of conveyance when conveying air, thus toward the catheter. For example, the rotational direction of a peristaltic pump can be reversed or the translational direction of a plunger of a displacement device, in particular a syringe, can be reversed.

Preferably, the fluid channel section comprises at least one inflow channel for supplying a liquid active agent, in particular a heparin solution or a beta blocker solution, into the fluid channel, preferably also—or additionally—into a second fluid channel. The inflow channel can be arranged in the housing of the fluid channel section and can open into an inlet connection which is arranged externally on the housing and which is designed to connect to a reservoir for the liquid active agent.

The inflow channel and/or the air outflow channel and/or the blood inflow channel and/or the blood outflow channel and/or the at least one fluid channel can in each case comprise at least one valve, in particular a check valve, in particular a non-return valve, by means of which a fluid only flows in one direction through the respective channel, and a check valve controllable by means of the control device, by means of which the fluid flow through the respective channel is permitted or blocked independent of direction, in particular can be metered, in order to particularly continuously or gradually change the cross section of the valve opening.

The non-return valve can in particular be a duckbill valve or a membrane check valve or ball check valve. The check valve can comprise an electrically driven valve actuator controllable by means of the control device.

It is particularly preferential for a check valve to comprise a pin movably supported on the fluid channel section, in particular its housing, the first end of which is disposed on the channel to be closed and the other end of which can be moved by an actuator device of the apparatus. If the channel is of elastically deformable design at the respective location, it can then be compressed by the pin and its opening cross section reduced and/or closed. The control device is to that end preferably designed to automatically control the actuator device in order to control the valve as predefined or, respectively, control the control program. The actuator device is in particular not a component part of the fluid channel section, which can thus in particular be particularly efficiently designed, including the pin, as a disposable item. The pin can, however, also be a moving part of the actuator device.

Preferably, the fluid channel section comprises a first valve, by means of which the air flow through the fluid channel can be varied. The first valve can be arranged on—or in—the air outflow channel. Preferably, the fluid channel section comprises a second valve, by means of which the rinsing fluid flow through the fluid channel can be varied. The second valve can be arranged on—or in—the rinsing inflow channel.

Preferably, the fluid channel section comprises a blood outflow channel in fluid connection with the fluid channel for delivering the blood from the fluid channel to an extracorporeal blood guidance system, and in particular a third valve, by means of which the fluid flow in the blood outflow channel can be varied. The third valve can be arranged on—or in—the blood outflow channel.

Preferably, the fluid channel section comprises: a second fluid channel and—a second blood inflow channel for connecting a second catheter to the second fluid channel to form a fluid connection between the second catheter and the second fluid channel. In hemodialysis, two connectors and two catheters are needed in order to connect the intracorporeal and the extracorporeal blood circulation. The connections necessary for the venting of these two catheters are preferably advantageously integrated into one (single) fluid channel section.

Preferably, the apparatus comprises a second sensor section which is designed to detect the entry of blood into the second fluid channel by means of a second sensor device and which is in particular—as is the first sensor section—integrated into the fluid channel section. The first and the second sensor section are preferably of identical configuration, although can also be of differing configuration. The first and the second sensor device are preferably of identical configuration, although can also be of differing configuration.

Preferably, the apparatus, in particular the fluid channel section, comprises a second sensor device which is designed to measure the deaerating and/or filling of the second fluid channel, in particular utilizing the second sensor section, and to produce a second measurement signal containing information on the deaerating and/or filling of the second fluid channel, whereby in particular the control device is designed to automatically control the deaeration device for deaerating and/or filling the second fluid channel as a function of the second measurement signal.

Preferably, an air outflow channel, in particular the one (and only) air outflow channel of the fluid channel section, is fluidly connected to the second fluid channel for its deaeration so that the air can be expelled out of the second fluid channel and out of the second catheter connected to the blood inflow channel through the air outflow channel by means of the deaeration device, whereby the second catheter connected to the blood vessel and the fluid channel can be filled with blood, wherein the fluid channel section in particular comprises a fourth valve, by means of which the air flow through the second fluid channel can be varied. The fourth valve can be arranged on—or in—the second fluid channel.

Preferably, the second fluid channel is fluidly connected in particular to the one (and only) rinsing inflow channel of the fluid channel section so that the physiological rinsing fluid can be fed into the second fluid channel and can flow through same, whereby the fluid channel section in particular comprises a fifth valve, by means of which the flow of the rinsing fluid through the second fluid channel can be varied. The fifth valve can be arranged on—or in—the second fluid channel.

The rinsing inflow channel provided for the deaeration of a catheter and the air outflow channel can be identical in that a channel through which matter can flow, in particular a fluid channel, can be used for the flow of air and for the flow of rinsing fluid in the direction of a catheter. The fluid channel section can exhibit a channel intersecting point, on one side of which opens a first and a second—flowable in both directions—fluid channel and on the other side of which opens the one (and only) rinsing inflow channel leading away from the one (and only) air outflow channel. This thereby enables efficiently producing the fluid channel section and enables the apparatus to operate it efficiently.

Preferably, the fluid channel section and/or in particular at least one catheter connected to the fluid channel section are designed as disposable items so as to ensure the sterility of the apparatus. Preferably, at least one catheter, in particular tube catheter, is connected to the fluid channel section, filled with a physiological rinsing fluid, in particular filled under sterile conditions and initially sealed in sterile manner, and in particular designed as a disposable item. The sterile filling can achieve a connection of the intracorporeal blood circulation with the fluid channel and in particular with the extracorporeal blood circulation which has a dearth of air bubbles, in particular requiring less or no deaerating effort.

Preferably, the deaeration device comprises a pump or a displacement device, in particular a syringe, wherein the displacement device in particular comprises a displacing plunger arranged in a hollow cylinder which draws the fluid into the fluid chamber of the hollow cylinder or drives it out of same through an opening in the hollow cylinder. The fluid chamber can be filled with a physiological rinsing fluid, in particular filled under sterile conditions and initially sealed in sterile manner.

Preferably, the apparatus comprises a reservoir containing the physiological rinsing fluid for filling the catheter which is filled in particular under sterile conditions and initially sealed in sterile manner and which comprises a connection channel by means of which the reservoir is or can be fluidly connected to the air outflow channel of the fluid channel section, wherein the control device and the deaeration device are in addition designed to

a) convey the air through the connection channel into the physiological rinsing fluid contained in the reservoir by means of the deaeration device to deaerate the catheter where it evacuates the connection channel by buoyancy and rises to the surface of the physiological rinsing fluid,

b) optionally convey the blood through the catheter and the fluid channel into the reservoir where it mixes with the physiological rinsing fluid contained in the reservoir,

c) and after the physiological rinsing fluid is deaerated, in particular by means of the deaeration device, convey it out of the reservoir through the connection channel and through the fluid channel section into the catheter.

Preferably, the deaeration device is formed by a displacement device, in particular a syringe, which comprises a displacing plunger arranged in a hollow cylinder which draws the fluid into the fluid chamber of the hollow cylinder through an opening in the hollow cylinder or drives it out of same through the opening, and wherein the reservoir is in particular formed by the fluid chamber of a displacement device.

The invention further relates to a cannulation robot for automatically cannulating the blood vessel of the patient with a cannula which comprises an apparatus in accordance with the invention and which is preferably designed to automatically deaerate and fill a catheter connected to the cannula with blood after the automatic cannulation of the blood vessel by means of the apparatus, in particular in preparation of hemodialysis.

The invention further relates to a system, in particular a dialysis system, comprising an extracorporeal system of channels and an apparatus in accordance with the invention, wherein the control device is preferably designed to automatically perform the deaerating and/or filling of the (at least one) catheter and the (at least one) fluid channel with blood and in particular automatically guide the blood after it enters into the fluid channel section into the extracorporeal channel system via the blood outflow channel.

The invention further relates to a method for the automatic deaerating and filling of a catheter connected to a blood vessel of a patient with blood, in particular in preparation of hemodialysis, comprising the steps:

Deaerating and filling of a fluid channel fluidly connected to the catheter by means of a deaeration device;

Detecting the deaerating and/or filling of the fluid channel by means of a sensor device which produces a measurement signal containing information on said deaerating and/or filling of the fluid channel;

Controlling the deaeration device as a function of the measurement signal by means of a control device comprising electrical circuitry in order to achieve the automated deaerating and/or filling of the fluid channel and the catheter.

To also particularly be considered as an invention is an inventive fluid channel section for guiding fluid flows during the automatic deaerating and filling of a catheter connected to a patient's blood vessel with blood, in particular in preparation of hemodialysis, which exhibits:
a blood inflow channel designed for the connection of the catheter and for a fluid connection with the fluid channel,
an air outflow channel designed for fluid connection with the fluid channel and the deaeration device so that air can be conveyed out of the fluid channel and out of the catheter connected to the blood inflow channel through the air outflow channel by means of the deaeration device, whereby the catheter connected to the blood vessel and the fluid channel can be filled with blood,
preferably: a sensor section designed to detect the entry of blood into the fluid channel by means of a sensor device;
preferably: the sensor device for detecting the entry of blood into the fluid channel;
preferably: a blood outflow channel designed to deliver the blood from the fluid channel to an extracorporeal blood guidance system;
preferably: a rinsing inflow channel designed for the supplying of and flowing through of the fluid channel with a physiological rinsing fluid;
preferably: a first valve with which the air flow through the fluid channel can be varied, and in particular a second valve with which the rinsing fluid flow through the fluid channel can be varied;
preferably: a blood outflow channel designed to deliver the blood from the fluid channel to an extracorporeal blood guidance system and which is fluidly connected to the fluid channel, and in particular a third valve with which the fluid flow in the blood outflow channel can be varied;
preferably: a second fluid channel and a second blood inflow channel for the connection of a second catheter to the second fluid channel to form a fluid connection of the second catheter to the second fluid channel;
preferably: a second sensor section designed to detect the entry of blood into the second fluid channel by means of a second sensor device;
preferably: a deaeration device for deaerating the (first) and in particular the second fluid channel;
preferably: the fluid channel section and/or in particular at least one catheter connected to the fluid channel section being designed as a disposable item;
preferably: wherein the air outflow channel is fluidly connected to the second fluid channel for its deaerating so that the air can be conveyed out of the second fluid channel and out of the second catheter connected to the blood inflow channel through the air outflow channel by means of the deaeration device, whereby the second catheter connected to the blood vessel and the fluid channel can be filled with blood, wherein the fluid channel section in particular exhibits a fourth valve with which the air flow through the second fluid channel can be varied, and wherein in particular the second fluid channel is fluidly connected to the rinsing inflow channel such that the physiological rinsing fluid can be supplied to and flow through the second fluid channel, whereby the fluid channel section in particular exhibits a fifth valve with which the rinsing fluid flow through the second fluid channel can be varied.

The fluid channel section of the inventive apparatus is preferably a fluid channel section in accordance with the invention. Further possible and preferential embodiments of the inventive fluid channel section can be derived from the description of the inventive apparatus and method and the embodiments of same.

The control device is preferably designed to achieve the deaerating and/or filling of the fluid channel by electronic control.

The puncturing of blood vessels, also known as cannulation, is a routine procedural step in the medical treatment of many patients in which a fluid connection, in particular a cannula, is established between a patient's blood circulation and an external fluid system. Cannulation is usually performed by physicians or trained personnel. The quality of the vascular access created by the cannulation thereby depends on a plurality of parameters, which are in particular affected by the individual and temporally varying abilities of the medical personnel and the physical characteristics of the patients to be treated as well as the diversity of the technical instruments used in cannulation.

Being a routine procedure in many treatments, cannulation is also frequently performed. In order to thereby standardize cannulation, make efficient use of financial as well as personnel resources, and reliably ensure high treatment quality, cannulation robots have been developed which autonomously perform a cannulation procedure on patients using suitable sensor technology and motor function. Such cannulation robots and the technical resources thereby used are known from WO 2010/029521 A2.

The inventive apparatus preferably comprises a base on which preferably all of the components of the apparatus are mounted, in particular the fluid channel section. The base can be a supporting device for supporting the extremities of the patient with the blood vessel to be punctured and the catheter to be deaerated. The base can be designed as an arm rest.

The apparatus preferably comprises a treatment chamber which can be a partly enclosed or open spatial area which can in particular be integrated into a treatment apparatus, in particular a cannulation robot. The treatment chamber serves to at least partly accommodate the part of the patient's body with the subcutaneous blood vessel. The body part is preferably an arm or a leg.

The treatment chamber preferably comprises a supporting device for supporting the body part, in particular a rest or a plurality of rests. Preferably, the treatment chamber comprises a fixation device, by means of which the patient's body part, in particular an arm or a leg, is immobilized in the treatment chamber.

Preferably, the control device is designed to perform the deaerating and/or filling and/or the one-time or repeated flushing of the fluid channel and the catheter within a predetermined time interval, in particular a time interval of less than 60 seconds, preferably less than 30 seconds, preferably less than 20 seconds, preferably less than 10 seconds, preferably less than 5 seconds.

Preferably, the apparatus according to the invention or one of its embodiments described herein is a component part of a treatment apparatus, in particular a cannulation robot. The cannulation robot, respectively the control device of the cannulation robot, is thereby preferably designed to automatically perform the cannulation of the blood vessel and in particular the deaerating and/or filing of the fluid channel. A treatment apparatus can further carry out a non-invasive treatment of the blood vessel.

The apparatus, respectively its control device, can be designed to access stored patient data—particularly a patient database—in order to determine information on past cannulation procedures. A cannulation robot can thereby be designed to determine suitable cannulation procedural steps in the cannulating of the patient's blood vessel from such patient data (historical data), and preferably determine the cannulation to be performed, in particular the program parameters used in the program-controlled automatic cannulation, as a function of said historical data. Such historical data contains in particular the position of one or more of the patient's skin sections and which is in particular available as patient data. Such historical data in particular contains information on the position and condition of prior puncture sites on the patient's body part which is in particular available as patient data.

In the sense of the invention, a “cannula” is a tubular body, in particular a rigid or flexible injection needle, having a lumen of a geometry and external dimensions suited for use in cannulation of a blood vessel. Preferably, the cannula comprises a hollow needle and a connector part.

The potential advantages as well as embodiments, further developments or variants of the invention cited previously also apply accordingly to the inventive cannulation robot.

A cannulation robot is an apparatus which automatically; i.e. at least intermittently or continuously, performs at least one cannulation process step in a patient blood vessel, or several or all intended process steps, without the intervention of a human operator, e.g. medical personnel. This thereby ensues in particular by the program parameters of the automated cannulation being accordingly selected by the system and/or by the user. One process step in the cannulation is in particular technically implemented by a component of the cannulation robot, e.g. a tool device, specifically configured for said process step and is selected from the group comprising the possible process steps P1, P2, P3 . . . , without this numbering defining a sequential ordering:
P1: Using an accessory kit to perform the cannulation which is selected prior to commencing the automated cannulation based on the registered patient identifier; this selection can have been made previously by means of an optional pick-and-place system of the system for selecting an accessory kit and/or equipping an accessory holder, in particular an accessory box; the accessory kit can have been provided beforehand as a function of the registered patient identifier by an optional sorting apparatus of the system selecting the accessories contained in the accessory kit from an optional storage apparatus of the system for storing accessories; the accessory kit can contain one or more medical accessories, in particular gauze, swabs, adhesive tape; the accessories of this accessory kit can be gathered as a function of the registered patient identifier and/or as a function of patient-specific treatment data derived from the registered patient identifier; the use of this accessory kit by the cannulation robot is a process step of the automated cannulation and can provide for the accessories of the accessory kit to be automatically extracted from predetermined positions of an accessory holder/box, in particular by the appropriate program parameters being selected as a function of the registered patient identifier and suitable for extraction; an optional pick-and-place device of the cannulation robot being in particular used to that end which is configured to extract the accessories out of the accessory holder and/or configured to equip one or more optional tool devices of the cannulation robot;
P2: Spatially fixating a part of the patient's body containing the blood vessel, in particular an arteriovenous fistula; the program parameters of the automated cannulation can be selected here as a function of the registered patient identifier, thus individual to each patient, these program parameters setting beforehand the position or the spacing of one or more optional fixation devices of the cannulation robot based on a previously determined location or on predetermined spacings on the patient's body part so as to achieve suitable fixation; the fixation taking place in the treatment chamber of the cannulation robot in which the patient's body part rests for the at least one ensuing cannulation;
P3: Using stored—in particular in a patient database—patient data in order to determine information on past cannulation process steps in the patient's vasculature (historical data), and preferably define the cannulation to occur, in particular the program parameters thereby used, based on this historical data; such historical data containing in particular the location of one or more of the patient's blood vessels previously measured by an optional measuring device of the cannulation robot for measuring the location and/or dimensions of at least one blood vessel under the patient's skin (vascular structure measuring device), and providing same in particular as patient data; such historical data containing in particular information on the location and condition of further puncture sites on the patient's body which is in particular provided as patient data; the vascular structure measuring device being able to be designed to detect the location and/or dimensions of at least one blood vessel under the patient's skin by means of ultrasound or by means of optical radiation;
P4: Identifying the blood vessel under the patient's skin suitable for the blood withdrawal, in particular selecting a suitable insertion site on the skin for the cannulation of said blood vessel; the program parameters of the automated cannulation can hereby be selected as a function of the registered patient identifier, thus individual to the specific patient, by the cannulation planned for the registered patient being selected on the basis of at least one patient-specific treatment parameter; for example with a patient planned for hemodialysis; a treatment parameter can encode the patient's necessity for hemodialysis; the cannulation of an arteriovenous blood vessel can be planned by evaluating the treatment parameter; same being identified; the identification can for example ensue in the control system by a program-controlled analysis of an image obtained by a vascular structure measuring device;
P5: Disinfecting the skin of the patient's body part containing the blood vessel; the program parameters of the automated cannulation can hereby be selected as a function of the registered patient identifier, thus individual to the patient, by a disinfecting process being specifically selected for the patient's type of skin or skin morphology which is for example characterized by the length of the treatment or the amount and nature of the disinfecting process employed; treatment data specific to the patient can also be considered; a disinfecting device which is optional with the cannulation robot or separate therefrom and equipped to perform the cited function can be used for the cited disinfection; the type of skin or skin morphology of the patient being preferably known in particular as patient data in the patient database;
P6: Physically treating the patient's body part containing the blood vessel in preparation for the cannulation, in particular stemming the blood flow of the body part, applying pressure to the body part, controlling the temperature of the body part, positioning the immobilized body part; the program parameters of the automated cannulation can hereby be selected as a function of the registered patient identifier, thus individual to the specific patient, by drawing on preparation data specific to the planned patient treatment, e.g. hemodialysis, or which can be taken from the patient database as known preparation data; this preparing for the cannulation of the body part being in particular performed by an optionally provided prepping device of the cannulation robot correspondingly configured for this purpose;
P7: Puncturing the blood vessel, in particular an arteriovenous fistula; preferably a first venipuncture and cannulation occurring automatically for withdrawing blood from the blood vessel and a second venipuncture and cannulation occurring automatically for the return of the blood, in particular in the case of hemodialysis; the program parameters of the automated cannulation can hereby be selected as a function of the registered patient identifier, thus individual to the specific patient, by the program parameters defining a patient-dependent motion control for a robotic tool arm optionally provided in the cannulation robot, by means of which a medical accessory such as for instance an injection needle can for example be grasped by the tool arm and positioned on the body part, with the injection needle having been previously selected and prepared specific to the patient; two cannulation robots can be set up for puncturing blood vessels at different parts of the body by, for example, a first cannulation robot being configured for cannulation on an arm and a second cannulation robot being configured for cannulation on a leg; the selection of the appropriate cannulation robot can ensue in patient-specific and/or treatment-specific manner;
P8: Withdrawing blood from the cannulated blood vessel and transporting the blood in at least one blood transport device or in at least one sample container; the program parameters of the automated cannulation can hereby be selected as a function of the registered patient identifier, thus individual to the specific patient, by a suitable blood transport device or suitable sample container being preselected as a function of patient-specific treatment data and then utilized in suitable manner by the cannulation robot; the cannulation robot and the control system can be configured thereto by an appropriate selection of the program parameters to provide at least one sample container based on treatment data for the subsequent, preferably automatic and system-controlled, treatment, in particular diagnostics;
P9: The grasping of a cannula by a gripper apparatus of the cannulation robot. The term “cannulation” refers to a procedure in which a cannula is inserted into the blood vessel in the patient's body part by puncturing the skin and venipuncturing the blood vessel wall so that the distal end of the cannula is disposed in the blood vessel and the proximal end of the cannula is disposed on the outside of the body part so that a fluid connection can be established between the cannula and the blood vessel, by means of which fluid, in particular blood and/or fluid media, can be exchanged via the fluid connection. The “exchange” of fluid in this context means that fluid from the patient's blood circulation is conveyed to an extracorporeal fluid system, i.e. situated external of the patient's body, in particular for fluid storage or fluid conduction, and/or includes conveying fluid from the extracorporeal system into the blood circulation.

Chronically ill patients need regularly repeated vasculature cannulation in order to ensure the necessary treatment. One such chronic illness is kidney failure which leads, among other things, to the loss of the blood's natural purifying function. Technical solutions can be substituted in its place. Hemodialysis devices are extracorporeal filtering units serving as artificial kidneys into which the blood of the patent is conducted in order to be cleansed and treated before being returned to the patient's blood circulation. Blood is normally withdrawn and returned via an artificial subcutaneous connection surgically created between a vein and an artery in an arm or a leg of the patient. This connection can be composed of a section of the patient's own vasculature prepared for same or can consist of an artificial material and is referred to as a fistula or arteriovenous fistula respectively (AV fistula, AVF).

The most commonly used permanent vascular access in chronic hemodialysis patients is a native arteriovenous fistula. After the native arteriovenous fistula is placed, it becomes stronger due to the increased blood flow, whereby repeated cannulation for the dialysis treatment becomes easier.

Hemodialysis must be performed regularly, typically a few days apart. This leads to high mechanical stress on the blood vessel or arteriovenous fistula respectively. Different techniques are known to create access to a blood vessel or arteriovenous fistula respectively, these aiming to be as gentle as possible on the vessel over the course of the repeated cannulation. In rope ladder cannulation, a new cannulation site located at a distance from the previous site, e.g. about 2 cm, is sought for each treatment. In this method, the series of punctures are usually started at the lower end of the vessel and then continue upward until reaching the upper end and the process then started again from below. The practitioner must thereby precisely follow the positioning pattern so as to allow the venipunctured vessel sites to heal. In contrast, in the buttonhole technique, a needle is always inserted into the exact same spot at the exact same angle. Over time, a scar tunnel thus develops which continually displaces the thrombus forming in cannulation and thus becomes more resilient. It has been found that buttonhole technique results can be improved if the cannulation is always performed by the same treatment personnel. For this reason, the use of a cannulation robot is particularly advantageous.

Due to the frequency of cannulation with hemodialysis patients, the arteriovenous fistula is subject in general to high stress, independent of the venipuncture technique, same which can lead to changes in the surface of the skin and the condition of the arteriovenous fistula and how they progress. The present invention allows regulated optimizing of the position and/or dimensions of the blood vessel so that in particular an automatic cannulation can be realized gently, quickly and efficiently.

One advantage of the cannulation robot with automatic disinfection machine can additionally be seen in that, in particular when treating chronic illnesses—in particular with hemodialysis patients—, the automated cannulation can reduce the workload of the medical personnel and/or provide a consistently high cannulation precision, whereby in particular treatment quality and/or treatment safety can be increased.

The potential advantages as well as embodiments, further developments or variants of the aspects of the invention cited previously also apply accordingly to the inventive method. Inversely, potential advantages as well as embodiments, further developments or variants of the method also apply accordingly to the preceding aspects of the invention.

As defined by the invention, “configured” refers to an apparatus not only being in principle suited to fulfill a specific function—for instance only after a specific program code has been loaded; i.e. the apparatus programmed, or the apparatus formed in a specific way—, but the apparatus already possesses all the means necessary in order to actually fulfill the function.

Preferably, the apparatus is to that end already programmed with a program code for said function and/or already configured and/or arranged and/or exhibits such a configuration thereto that the apparatus actually fulfills the function.

“Treatment of a patient” in the sense of the invention refers to at least one medical; i.e. in particular therapeutic, diagnostic or cosmetic, procedure which effects changes to the body and/or health of the patient or by means of which the state of the patient's health is determined. A treatment is in particular an administration of medicinal products, a cannulation, a blood purification procedure such as dialysis, an operation and/or an examination of the patient.

A “group of treatments” in the sense of the invention can be respective specific operations, therapy for a specific illness, the initial examination of a patient, or a dialysis treatment which in turn can comprise sub-groups, in particular hemodialysis, hemofil-tration, hemodiafiltration, hemoperfusion or peritoneal dialysis treatments.

As defined by the invention, an “individual involved in the treatment” can in particular be understood as an attending person, for instance a physician, or an individual providing treatment support, for instance a nurse. In particular, the patient to be treated can himself also be an individual involved in the treatment or an attending person.

A control device of the detection device and/or the cannulation robot preferably comprises a data processing apparatus and is thus preferably a data processing control device.

To be understood by a “data processing apparatus” is an apparatus configured to process data; i.e. in particular to receive data, store received data, read out stored data, transform received and/or stored and/or read data by means of logical and/or mathematical operations, store transformed data, and/or output transformed and/or read data. Preferably, such a data processing device is programmable; i.e. a program code in particular at least partially specifies the method for processing the data and at least part of said program code is modifiable. The program code can to that end be configured to automatically control the apparatus and/or the deaeration device or conveyor device and/or evaluate the measurement signal data.

Preferably, the data processing apparatus is a commercially available microprocessor or computer. Further preferentially, the data processing apparatus comprises at least one data processor—i.e. a central processing unit—, a non-volatile—i.e. in particular permanent—data storage, in particular a hard disk, a read-only memory (ROM) or a drive with a data medium, as well as at least one hardware interface. The data processing apparatus also preferably comprises a volatile electrical data storage, in particular as main memory, preferably a semiconductor memory, in particular with integrated capacitors and/or flip-flops (bistable multivibrators) for data storage, for instance dynamic RAM or static RAM.

In the sense of the invention, a “data storage apparatus” or “data storage device” is an apparatus for storing data. Same is in particular designed to form a data link with a further apparatus, particularly a data processing apparatus, and/or comprises a data link to the further apparatus, wherein data can be transmitted to the data storage apparatus from the further apparatus for storage by means of the data link and/or data can be transmitted from the data storage apparatus to the further apparatus for retrieval. Preferably, the data storage apparatus comprises at least one non-volatile data storage. Also preferably, the data storage apparatus comprises at least one volatile electrical data storage.

A data link connects in particular two data processing units, in particular two data processing devices or apparatus, in a way so as to enable the exchange of data between the units, either unidirectionally or bidirectionally. The data link can be realized in wired or wireless manner, in particular as a radio link. A remote data link connects in particular two data processing units, particularly two data processing devices, disposed at a distance from one another, thus not being component parts of the same device, in particular the same user interface device or the same control system, if the cited devices are realized as separate units. A data link, in particular remote data link, of one device to another device is preferably realized by a direct connection between the two devices or by an indirect connection of the two devices such that a third device is connected between the two devices in order to pass on the data. A remote data link can in particular be realized by a network of computers with which the devices connected by the remote data link are interconnected via the network. The network can be a restricted network, e.g. an intranet, or global network, in particular a WAN and/or the internet.

In the sense of the invention, an “interface device” serves the connection of two units—in particular including systems, apparatus, devices or mechanisms, particularly having such units—, respectively capable of processing signals, in particular information, particularly data, thus in particular sending and/or receiving. An interface device can comprise at least one hardware interface and in particular be integrated into a physical device unit as a component part.

The term “treatment of a laboratory sample” in particular means that a laboratory sample, in particular a sample or a volume of blood, is moved and/or transported and/or examined and/or physically, chemically, biochemically or in some other way modified, in particular as regards its composition. The inventive apparatus can in particular be employed to acquire a laboratory sample, whereby the blood output via a blood outflow channel is in particular conducted into a sample container.

Preferably, the apparatus according to the invention, in particular a treatment apparatus comprising the inventive apparatus, comprises at least one of the following components: a user interface device, with which a user can make at least one data input which is processed by the control device or its program code, and/or with which information can be output to the user, wherein the user interface device can comprise a display, in particular a touchscreen, speaker and/or input device such as e.g. a keyboard; a housing; a recording device for recording the measurement signals particularly as a function of time data; a base, in particular having a supporting frame bearing the component parts of the apparatus or at least one of said components; a power supply system for supplying power to the electrical components of the apparatus; a communication device for exchanging data with an external data processing apparatus, in particular via remote data link.

The invention further relates to the system comprising an apparatus according to the present description and/or a treatment apparatus comprising said apparatus, in particular a cannulation robot, and at least one external data processing apparatus linked to the apparatus and/or the treatment apparatus for the exchange of data, in particular via a data link or a remote data link. The system can further comprise a data storage apparatus as a component, same being linked to at least one other system component for exchanging data. The data storage apparatus can contain a patient database in which patient data is stored and able to be retrieved. The system can to that end be designed to acquire and store certificate data, in particular save it to the data storage apparatus. The certificate data can in particular encompass measurement signals and/or thereby in particular correlated time data.

The invention further relates to a method for the automatic cannulation of a patient's blood vessel, in particular a method for operating a cannulation robot, particularly a cannulation robot according to the invention, comprising the steps of the inventive method for automatically deaerating and/or filling the catheter, and comprising the step(s) of: Automatic cannulation of the blood vessel; optionally: Performing said cannulation and catheter deaeration and/or filling within a predetermined limited interval of time.

Further advantages, features and possible applications of the present invention are yielded by the following detailed description of at least one example embodiment and/or by the figures. Unless otherwise described or contextually indicated otherwise, the same reference numerals are substantially used to identify equivalent components in the embodiments. The figures show the following example embodiments of the invention:

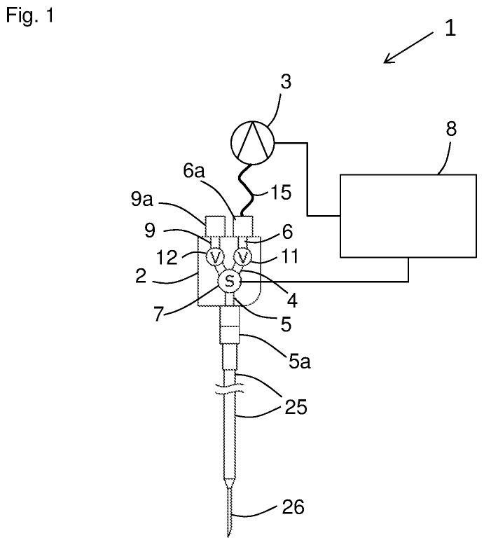

FIG. 1 shows a schematic view of an inventive apparatus according to a first example embodiment.

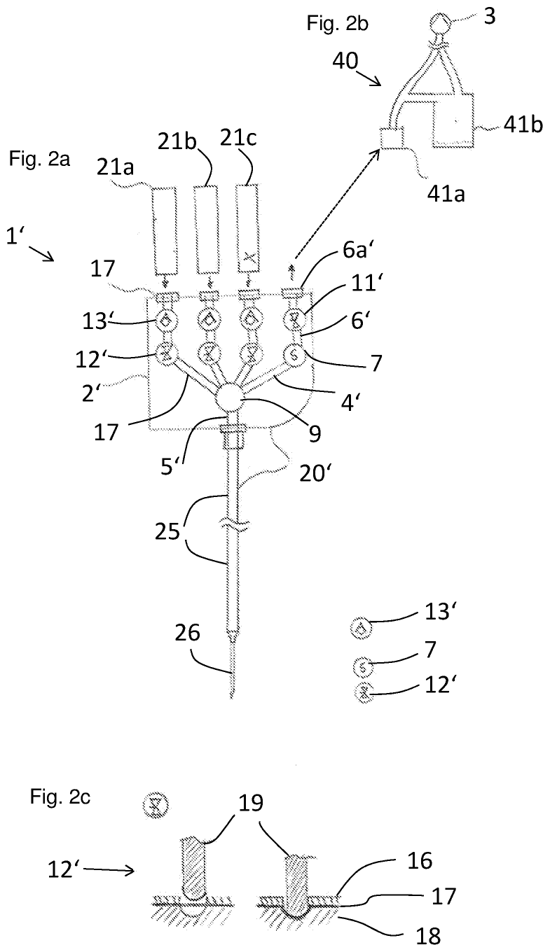

FIG. 2a shows a schematic view of an inventive apparatus according to a second example embodiment.

FIG. 2b shows a schematic view of an overfill protection for use with the apparatus of FIG. 2a.

FIG. 2c shows a schematic view of a check valve for use with the apparatus of FIG. 2a.

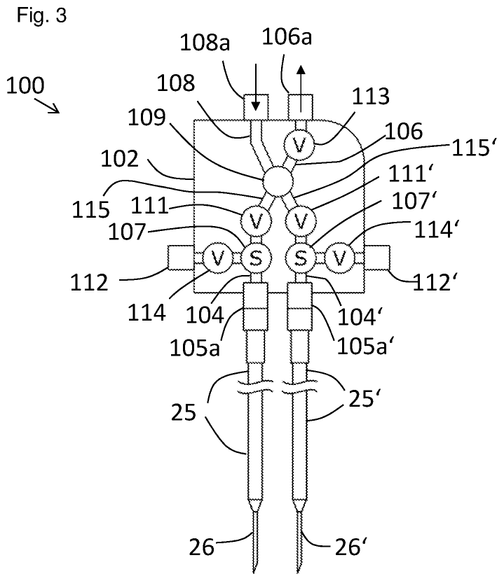

FIG. 3 shows a schematic view of an inventive apparatus according to a third example embodiment.

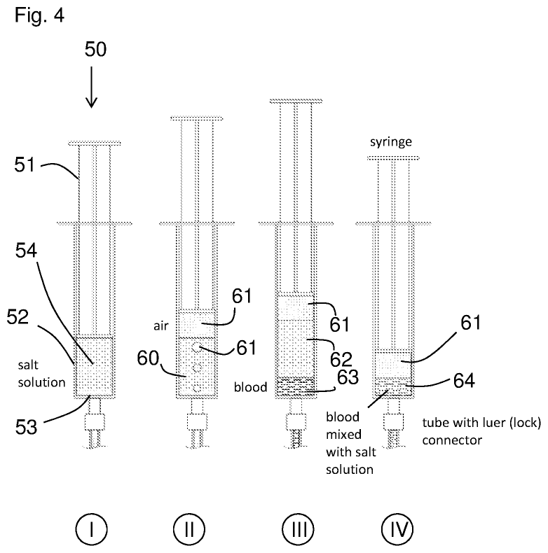

FIG. 4 shows a schematic view of a combined deaeration and flushing system for use with the apparatus of FIG. 1, 2a or 3, in four different phases.

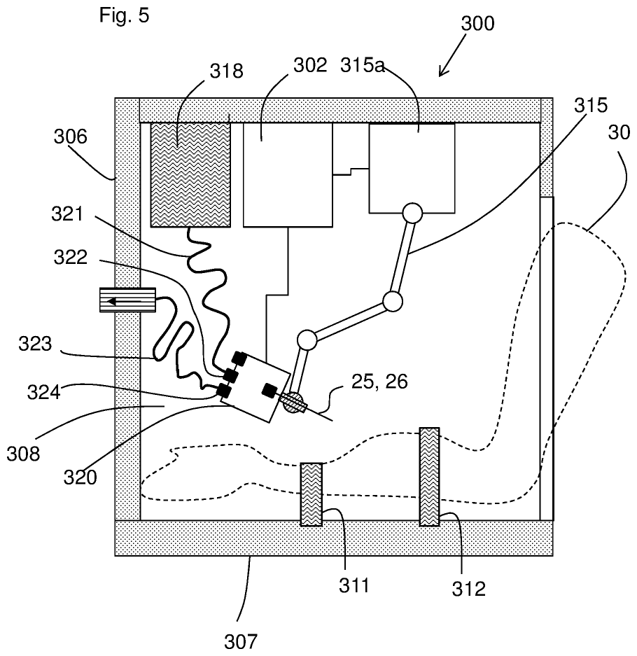

FIG. 5 shows a schematic view of the example embodiment of a cannulation robot comprising an apparatus according to the invention.

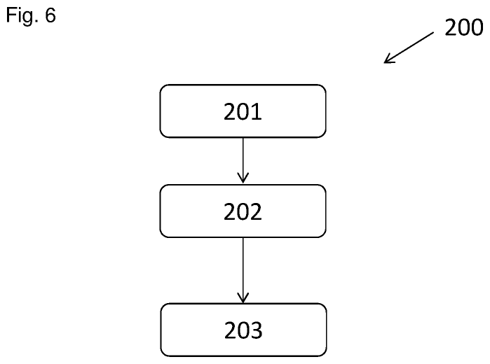

FIG. 6 shows a schematic view of the example embodiment of an inventive method for the automatic deaerating and filling of a catheter connected to a blood vessel of a patient.

FIG. 1 shows an apparatus 1 for the automatic deaerating and filling of a catheter 25 and a cannula 26 connected to a blood vessel of a patient with blood. The apparatus is used in preparation for hemodialysis. The apparatus comprises: a fluid channel section 2 for guiding fluid flows, a deaeration device 3 for deaerating the catheter, wherein the fluid channel section comprises: a fluid channel 4 through which fluids can flow, a blood inflow channel 5 configured for the catheter attachment and for a fluid connection to the fluid channel, an air outflow channel 6 configured for a fluid connection to the fluid channel and the deaeration device such that air can be conveyed out of the fluid channel and out of the catheter connected to the blood inflow channel through the air outflow channel by means of the deaeration device, whereby the catheter connected to the blood vessel and the fluid channel can be filled with blood. The apparatus thereby comprises a sensor device 7 designed to measure the filling of the fluid channel and to generate a measurement signal containing information on the filling of the fluid channel. The apparatus 1 further comprises a control device 8 with electrical circuitry which is configured to automatically control the deaeration device 3 for deaerating and/or filling the fluid channel as a function of the measurement signal.

A sensor device 7 is arranged at sensor section “S” of fluid channel section 2. The control device 8 is connected to the sensor device 5 in order to receive the measurement signal. The control device 8 is also connected to the deaeration device 3, here a peristaltic pump, in order to induce by means of its control the conveyance of the air out of the air outflow channel 6 via the channel connector 6a and via the air duct 15 to the deaeration device.

The fluid channel section 2 here additionally comprises an air outflow channel 6 with a channel connector 6a. The valve 11 is a check valve designed to block or open the fluid channel 4 so as to enable, prevent or respectively regulate—as controlled by the control device 8—the passage of air through the fluid channel 4 toward the deaeration device 3. The valve 11 serves as a first valve by means of which the air flow through the fluid channel can be varied.

The fluid channel section 2 here additionally comprises a blood outflow channel 9 with a channel connector 9a. The valve 12 is a check valve designed to block or open the blood outflow channel 9 so as to enable, prevent or respectively regulate—as controlled by the control device 8—the passage of blood in the direction of the channel connector 9a. The valve 12 serves as a third valve by means of which the air flow through the fluid channel can be varied.

The channel connectors 5a, 6a and 9a are in particular luer lock connections.

FIG. 2a shows an apparatus 1′ for the automatic deaerating and filling of a catheter 25 and a cannula 26 connected to a blood vessel of a patient with blood, wherein the apparatus is used in preparation for hemodialysis. The apparatus comprises: a fluid channel section 2′ for guiding fluid flows, a deaeration device 3 (see FIG. 1 or 2b) for deaerating the catheter, wherein the fluid channel section comprises: a fluid channel 4′ through which fluids can flow, a blood inflow channel 5 configured for catheter attachment and for a fluid connection to the fluid channel 4′, an air outflow channel 6′ configured for a fluid connection to the fluid channel and the deaeration device such that air can be conveyed out of the fluid channel 4′ and out of the catheter 25 connected to the blood inflow channel through the air outflow channel 6′ by means of the deaeration device 3, whereby the catheter connected to the blood vessel and the fluid channel can be filled with blood. The apparatus thereby comprises a sensor device 7, in particular an optical measuring device, designed to measure the filling of the fluid channel and to generate a measurement signal containing information on the filling of the fluid channel. The apparatus 1′ further comprises a control device 8 (not shown) with electrical circuitry which is configured to automatically control the deaeration device 3 for deaerating and/or filling the fluid channel as a function of the measurement signal. The control by means of the control device 8 preferably ensues such that the entry of the blood into the measuring zone of sensor 7 is detected during the deaeration by the deaeration device 3 and the deaeration device 3 is immediately stopped upon such entry being detected so that no further air is conveyed and the control device 8 closes check valve 11′. In this state, physiological rinsing fluid stored in reservoir 21c can be introduced into the fluid channel 4′ via the channel intersecting point 9 where the rinsing fluid mixes with the blood drawn into the fluid channel 4′. In particular, a conveyor device, in particular a pump, e.g. a peristaltic pump, can be provided in the channel intersecting point 9 or in another channel 17 or in a reservoir 21a, 21b, 21c with which the fluid can be pumped from reservoir 21a, 21b, 21c on the condition that the channel 17 blockable there by means of the non-return valve 13′ and check valve 12′ is respectively open. Preferably the control device is hereby designed to open the check valve 12′ as needed in order to simultaneously or during the same period feed the rinsing fluid, a first drug solution—e.g. heparin—and a second drug solution—e.g. a beta blocker—as needed into the fluid channel 4′ where the respective fluids mix with the blood contained in the fluid channel 4′.

FIG. 2b shows a connection possibility between the deaeration device 3 and the channel connector 6a′ of the fluid channel section. A channel connector 41a, which can in particular be connected to channel connector 6a′ via a luer lock connection, is thereby connected to the deaeration device by a channel, whereby a bypass channel with overfill protection reservoir 41b is provided in which any liquid possibly infiltrating through the connector 41a is collected. This thereby protects the deaeration device, which can comprise a vacuum pump, from entry of liquid.

FIG. 2c shows an example embodiment of a check valve which can be used in the apparatus according to the invention. The check valve comprises a pin 19, by means of which an elastically deformable tube channel 17 can be compressed and thereby closed. In the image on the left, the pin is not deflected, in the image on the right, it is deflected in order to block the channel.

Alternatively, the fluid channel section 2′ in FIG. 2c can comprise an upper plate section 16 with an opening for the pin and a lower plate section 18 defining a depression covered by an elastically deformable membrane arranged between the upper and lower plate section. The pin presses the membrane downward in a closure portion of the channel to block the channel formed by the depression and the membrane. The pin can be deflected by means of an electrically driven actuator device controlled by the control device.

FIG. 3 shows an apparatus 100 for the automatic deaerating, filling and flushing of a first catheter 25 and cannula 26 connected to a blood vessel of a patient as well as for the automatic deaerating, filling and flushing of a second catheter 25′ and cannula 26′ connected to another blood vessel of a patient with blood. After the deaerating, filling and flushing of the first and second catheter—also called “priming” the catheter—the blood is cleansed by an extracorporeal dialysis device (not shown). The apparatus 100 comprises: a fluid channel section 102 for guiding fluid flows, a deaeration device 3 (see FIG. 1 or 2b) for connecting to the channel connector 106a of the air outflow channel 106, a rinsing inflow channel 108 through which fluid with rinsing fluid which can be conveyed by a conveying device, in particular a pump (not shown), can flow from a reservoir filled with rinsing fluid via the channel connector 108a. The entry of blood into the measuring zone “S” of the fluid channel section is in each case detected by means of the sensor devices 107, 107′ which can comprise optical sensors.

The rinsing fluid can be conveyed into the first fluid channel 104 and the second fluid channel 104′ simultaneously or at staggered intervals. The fluid flow of the air between the first fluid channel 104 and the air outflow channel 106 can be controlled by valve 111 arranged at the first fluid channel 104, and the fluid flow of the rinsing fluid between the rinsing inflow channel 108 and the first fluid channel 104 can additionally be controlled by means of the control device. The fluid flow of the air between the first fluid channel 104′ and the air outflow channel 106 can additionally be controlled by valve 111′ arranged at the second fluid channel 104′ and the control device can additionally control the fluid flow of the rinsing fluid between the rinsing inflow channel 108 and the second fluid channel 104′. A check valve 113 serves in closing the air outflow channel 106, controlled by the control device. Valve 114 closes off the blood outflow channel running from the first fluid channel 104 and opening into the channel connector 112, valve 114′ closes off the blood outflow channel running from the second fluid channel 104′ and opening into the channel connector 112′. The rinsing inflow channel and air outflow channel 115, 115′ provided for the deaerating of each catheter are identical here in that a channel 115, 115′ through which matter can flow in both directions is used for the flowing of the air and for the flowing of the rinsing fluid toward the catheter. The fluid channel section exhibits a channel intersecting point 109, on one side of which opens the first 115 and the second 115′ fluid channels—through which fluid can flow in both directions—and on the other side of which opens the one (and only) rinsing inflow channel 108 leading away from the one (and only) air outflow channel 106. This thereby enables efficiently producing the fluid channel section 102 and enables the apparatus 100 to operate it efficiently.

FIG. 4 shows a schematic view of a combined deaeration and flushing device 50 for use with the apparatus in FIG. 1, 2a or 3 in four different phases which are respectively controlled by the control device controlling the deflection of the plunger 51. The deaeration and flushing device 50 is a displacement device 50, in particular a syringe 50, having a displacing plunger 51 which is movable inside a hollow cylinder 52 and arranged so as to be able to be driven by an actuator device (not shown) of the apparatus. The syringe 50 is a reservoir filled under sterile conditions which is automatically loaded by the apparatus or manually loaded into the connecting position in which the opening port 53 is connected to the channel connector 6a or 6a′ for the syringe disposed with a sterile physiological rinsing fluid. In the second phase, to deaerate the fluid channel and the catheter, the plunger 51 is deflected in order to enlarge the fluid chamber 54. The air 61 thereby passes through the opening port 53 of the syringe into the fluid chamber in Phase II and pushes upward through the rinsing fluid 60 due to the density differences between the air and the rinsing fluid, the air thereby separated from the opening port 53. Deaeration here does not yet stop at that moment of the blood reaching the measuring zone S of the sensor device but rather a further predetermined volume of the blood is further conveyed in Phase III such that a specific volume of blood 63 reaches the rinsing fluid in the fluid chamber of the syringe 54. In Phase IV, the mixture of rinsing fluid and blood is returned to the fluid channel and the catheter in order to flush them. The movement of the plunger and thus the flushing process can be repeated as needed.

FIG. 5 shows a schematic view of the example embodiment of a cannulation robot 300 comprising an apparatus 1, 1′, 100 according to the invention. The arm of a patient is positioned in the treatment chamber 308 and immobilized there by means of the fixation straps 311, 312. The components of the apparatus are supported by a base 307 which in particular comprises a supporting frame 306.

The cannulation robot 300 serves in the automatic cannulation of the patient's blood vessel with a cannula and comprises an apparatus 1, 1′ or 100. The cannulation robot is thereby configured to perform the automatic deaerating and filling of a catheter connected to the cannula with blood, in particular in preparation for hemodialysis, after the automatic cannulation of the blood vessel by means of said apparatus 1, 1′, 100. The cannulation robot 300 comprises a control and drive device 315a, by means of which a movable robotic arm 315 of the cannulation robot can be driven and controlled. The control device 2 of the cannulation robot here comprises the control device of the inventive apparatus and is electrically connected to the deaeration device (not visible) integrated here into the fluid channel section, the sensor device (not visible) arranged at the fluid channel section and the check valves (not visible) integrated into the fluid channel section, in particular in order to receive the measurement signal from the sensor device. The fluid channel section 320 of the inventive apparatus is connected here to the tool head which—controlled by the robotic arm 315—positions the cannula 26 connected to the catheter 25 for the automated cannulating of the blood vessel. Rinsing fluid is conveyed from the storage reservoir 318 of the cannulation robot through flushing tube 321 via the channel connector 322 of fluid channel section 320 for flushing into the fluid channel (not visible) of fluid channel section 320, the blood is further conveyed through the blood tube 323 to an extracorporeal dialysis system via channel connector 324 of the fluid channel section 320. The dialysis system comprises an extracorporeal channel system and an inventive cannulation robot 300, wherein the control device 2 is thereby designed to automatically perform the deaeration and/or filling of the catheter and at least one fluid channel with blood and to automatically guide the blood into the extracorporeal channel system via the blood outflow channel after it enters into the fluid channel section.

FIG. 6 shows a schematic view of the example embodiment of an inventive method 200 for the automatic deaerating and filling of a catheter connected to a blood vessel of a patient, comprising the steps:

Deaerating and filling a fluid channel fluidly connected to the catheter by means of a deaeration device; (201)

Detecting the deaerating and/or filling of the fluid channel by means of a sensor device which produces a measurement signal containing information on said deaerating and/or filling of the fluid channel; (202)

Controlling the deaeration device as a function of the measurement signal by means of a control device having electrical circuitry in order to achieve the automated deaerating and/or filling of the fluid channel and the catheter. (203)

1. An apparatus for the automatic deaerating and filling of a catheter connected to a blood vessel of a patient with blood comprising
a fluid channel section for guiding fluid flows,
a deaeration device for deaerating the catheter,
wherein the fluid channel section comprises:
at least one fluid channel through which fluids can flow,
a blood inflow channel configured for catheter attachment and for a fluid connection to the fluid channel,
an air outflow channel configured for a fluid connection to the fluid channel and the deaeration device such that air can be conveyed out of the fluid channel and out of the catheter connected to the blood inflow channel through the air outflow channel by means of the deaeration device, whereby the catheter connected to the blood vessel and the fluid channel can be filled with blood,
wherein the apparatus comprises a sensor device designed to measure the deaerating and/or filling of the fluid channel and to generate a measurement signal containing information on the deaerating and/or filling of the fluid channel, and wherein the apparatus comprises a control device having electrical circuitry which is configured to automatically control the deaeration device for deaerating and/or filling the fluid channel as a function of the measurement signal.

2. The apparatus according to claim 1 comprising a blood outflow channel designed to deliver the blood from the fluid channel to an extracorporeal blood guidance system.

3. The apparatus according to claim 1, wherein the fluid channel section comprises a sensor section S designed to detect the entry of blood into the fluid channel by means of the sensor device.

4. The apparatus according to claim 1, wherein the sensor device and/or the deaeration device is/are a part of the fluid channel section.

5. The apparatus according to claim 1, wherein the fluid channel section comprises a rinsing inflow channel designed for the supplying and the flowing of a physiological rinsing fluid through said fluid channel.

6. The apparatus according to claim 5, wherein the fluid channel section comprises a first valve, by means of which the air flow through the fluid channel can be varied, and a second valve, by means of which the rinsing fluid flow through the fluid channel can be varied.

7. The apparatus according to claim 5, wherein the fluid channel section comprises a blood outflow channel (9 in fluid connection with the fluid channel for delivering the blood from the fluid channel to an extracorporeal blood guidance system, and a third valve, by means of which the fluid flow in the blood outflow channel can be varied.

8. The apparatus according to claims 5, wherein the fluid channel section comprises:
a second fluid channel and
a second blood inflow channel for connecting a second catheter to the second fluid channel to form a fluid connection between the second catheter and the second fluid channel.

9. The apparatus according to claim 8, comprising a second sensor section S designed to detect the entry of blood into the second fluid channel by means of a second sensor device.

10. The apparatus according to claim 8, comprising a second sensor device designed to measure the deaerating and/or filling of the second fluid channel, utilizing the second sensor section, and to produce a second measurement signal containing information on the deaerating and/or filling of the second fluid channel, wherein the control device is designed to automatically control the deaeration device for deaerating and/or filling the second fluid channel as a function of the second measurement signal.

11. The apparatus according to claim 5, wherein the air outflow channel is fluidly connected to the second fluid channel for the deaeration of same such that air can be conveyed out of the second fluid channel and out of the second catheter connected to the blood inflow channel through the air outflow channel by means of the deaeration device, whereby the second catheter connected to the blood vessel and the fluid channel can be filled with blood, wherein the fluid channel section exhibits a fourth valve, by means of which the air flow through the second fluid channel can be varied,
wherein the second fluid channel is fluidly connected to the rinsing inflow channel such that the physiological rinsing fluid can be supplied to and flow through the second fluid channel, wherein the fluid channel section exhibits a fifth valve, by means of which the rinsing fluid flow through the second fluid channel can be varied.

12. The apparatus according to claim 1, wherein the fluid channel section, and/or at least one catheter connected to the fluid channel section, are designed as disposable items.

13. The apparatus according to claim 1, wherein the deaeration device comprises a pump or a displacement device wherein the displacement device comprises a displacing plunger arranged in a hollow cylinder which draws the fluid into the fluid chamber of the hollow cylinder or drives it out of same through an opening in the hollow cylinder.

14. The apparatus according to claim 1, wherein the apparatus comprises a reservoir containing the physiological rinsing fluid for filling the catheter and which comprises a connection channel by means of which the reservoir is fluidly connected to the air outflow channel of the fluid channel section, wherein the control device and the deaeration device are thereto designed to
a) convey the air through the connection channel into the physiological rinsing fluid contained in the reservoir by means of the deaeration device to deaerate the catheter where it evacuates the connection channel by buoyancy and rises to the surface of the physiological rinsing fluid,
b) optionally convey the blood through the catheter and the fluid channel into the reservoir where it mixes with the physiological rinsing fluid contained in the reservoir,
c) and after the physiological rinsing fluid is deaerated, convey it out of the reservoir through the connection channel and through the fluid channel section into the catheter.

15. The apparatus according to claim 14, wherein the deaeration device is formed by a displacement device which comprises a displacing plunger arranged in a hollow cylinder which draws the fluid into the fluid chamber of the hollow cylinder through an opening in the hollow cylinder or drives it out of same through the opening, and wherein the reservoir is formed by the fluid chamber of a displacement device.

16. A cannulation robot for automatically cannulating the blood vessel of the patient with a cannula which comprises an apparatus according to claim 1, designed to automatically deaerate and fill a catheter connected to the cannula with blood after the automatic cannulation of the blood vessel by means of the apparatus, in particular in preparation for hemodialysis.

17. A system comprising an extracorporeal system of channels and an apparatus according to claim 1, wherein the control device is designed to automatically perform the deaerating and/or filling of the catheter and at least one fluid channel with blood and automatically guide the blood after it enters into the fluid channel section into the extracorporeal channel system via the blood outflow channel.

18. A method for the automatic deaerating and filling of a catheter connected to a blood vessel of a patient with blood in preparation for hemodialysis, comprising the steps:
Deaerating and filling of a fluid channel fluidly connected to the catheter by means of a deaeration device;
Detecting the deaerating and/or filling of the fluid channel by means of a sensor device which produces a measurement signal containing information on said deaerating and/or filling of the fluid channel;
Controlling the deaeration device as a function of the measurement signal by means of a control device with electrical circuitry so as to achieve the automated deaerating and/or filling of the fluid channel and the catheter.
